(19) United States
(12) Patent Application Publication
(10) Pub. No.: US 2015/0038674 A1
(43) Pub. Date: Feb. 5, 2015

(54) USE OF GLP-2 ANALOGUES IN PULMONARY DISEASES FOR THERAPEUTIC PURPOSE

(71) Applicants: Bertan Boran BAYRAK, Istanbul (TR); Fusun OZTAY, Istanbul (TR); Sehnaz BOLKENT, Istanbul (TR); B. Pelin Arda PIRINCCI, Istanbul (TR); Refiye YANARDAG, Istanbul (TR)

(72) Inventors: B. Pelin Arda PIRINCCI, ISTANBUL (TR); Sehnaz BOLKENT, Istanbul (TR); Fusun OZTAY, Istanbul (TR); Refiye YANARDAG, Istanbul (TR); Bertan Boran BAYRAK, Istanbul (TR)

(21) Appl. No.: 14/065,401

(22) Filed: Oct. 28, 2013

(30) Foreign Application Priority Data
Aug. 5, 2013 (TR) 2013/09495

(51) Int. Cl. A61K 38/26

(52) U.S. Cl. CPC A61K 38/26
USPC 530/324

(57) ABSTRACT

The present invention relates to the use of GLP-2 analogue in an efficient amount for the production of a drug specific to treat a pulmonary disease which IS caused by oxidative stress, inflammation and/or apoptosis in an organism.

### FIELD OF THE INVENTION

The present invention relates to use of GLP-2 analogues also including teduglutide in acute and chronic pulmonary diseases associated with apoptosis, oxidative stress and inflammation for protection and therapeutic purposes.

### BACKGROUND OF THE INVENTION

Glucagon-like peptide-2 (GLP-2) is a proglucagon-derived peptide hormone which is secreted from endocrine L cells in the intestine. It is stated that GLP-2 increases the absorption of food in the intestine and improves the intestinal adaptation which deteriorates as a result of the disease in case large part of small intestine is removed from the body in experimental animals and in humans with short bowel syndrome (Jeppesen et.al., 2001). Experimental studies carried out in the recent years suggest that GLP-2 exhibits trophic effect specific to small and large intestine by stimulating the proliferation of epithelial cells and by enabling proteolysis inhibition and apoptosis (Drucker et.al, 1996, Estal and Drucker, 2003, 2005). It was shown that GLP-2 given as exogen in various experimnental models such as small bowel enteritis produced via chemicals (Boushey et.al., 2001), vascular ischenia/reperfusion damage (Prasad et.al., 2000), colitis induced by dextrane sulfate (L'heureux and Brubaker, 2003), or its destruction resistant analogues are associated with reducing epithelial damage, bacterial infections and mortality.

In clinical studies carried out based on the experimental data mentioned above, the usability of GLP-2 or protease resistant analogues of GLP-2 for therapeutic purposes was tested in inflammatory bowel diseases, patients with short bowel syndrome, intestinal damage seen as a result of chemotherapy or radiation treatment, systemic infections originating from organisms in the gastrointestinal tract and in cases wherein the food absorption in the intestine is deteriorated. Jeppesen et.al. have reported that subcutaneous administration of human GLP-2 in 11 patients with short bowel syndrome 3 times a day in 0.4 mg doses of 13, 26, 52 weeks does not change intestinal morphology, absorption, renal functions, bone mineral density and muscle functions (Jeppesen et.al., 2009). In another clinical study carried out to improve liver and kidney dysfunctions associated with parenteral nutrition and intestinal damage in patients with short bowel syndrome, it is stated that teduglutide delivered subcutaneously in 0.05 or 0.10 mg/kg/day doses has caused improvement in liver function tests such as AST (aspartate aminotransferase), ALT (alanine aminotransferase), ALP (alkali phosphatase), and renal function tests such as creatinine, urea, bilirubin and glomerular filtration speed (WO 2011/143335 A2). It was shown that teduglutide administered subcutaneously in doses of 0.05 or 0.10 mg/kg/day to 52 patients having intestinal damage with short bowel syndrome for 52 weeks is reliable for long term use in humans. The most common adverse effects seen in long term teduglutide use are headache (35%), nausea (31%), and abdominal pain (25%) (O'keefe et.al., 2013).

GLP-2 shows its effect via a specific receptor associated with G protein having 7 transmembrane areas belonging to glucagon-secretin receptor family. Yusta et. al. (2000) have identified the presence of GLP-2 receptor mRNA transcripts in rodents and humans via Nothern blotting and RT-PCR in stomach, duodenum, jejunum, ileum, colon, hypothalamus, brainstem and lung homogenates. In addition, it was shown that GLP-2 receptor (GLP-2R) is present in endocrine cells in the human stomach and intestine (Yusta et.al., 2000), in neurons in the intestines of rodents (Bjerkenes and Cheng, 2001), in specific areas of central nervous system (Lovshin et.al., 2001), in subepithelial myofibroblasts (Orskov et al., 2005), pancreatic alpha cells of hmnans and rats (de Heer et al., 2007), and in rat heart (Angelone et al., 2012).

However, the effects of GLP-2 on healthy and pathologic lung have not been searched yet.

Pulmonary epithelial and endothelial damages play an important role in acute lung injuries and some mortal chronic lung diseases. Pulmonary epithelial and endothelial damages are very importance since these areas are where the oxygen and carbon dioxide exchange between inspiration air and the blood takes place. This situation can result in respiratory failure depending on the degree of damage. Pulmonary epithelial and endothelial damage, and various pathological symptoms followed the damage are reported in other interstitial pulmonary diseases such as acute lung damage, acute respiratory failure syndrome, chronic obstructive pulmonary disease, emphysema, pulmonary hypertension and pulmonary fibrosis.

Acute lung damage can easily be stimulated by aspiration, pneumonia, systemic sepsis, shock, cardiopulmonary by-pass. Chronic obstructive pulmonary disease (COPD) is shown as the 4^{th} cause of death in developed countries in the world. Generally chronic obstructive pulmonary disease is accompanied with emphysema. Even though pulmonary fibrosis is not as common as chronic obstructive pulmonary disease, it is a highly aggressive disease in terms of progression of disease and being mortal. Average lifetime is 3-5 years in patients with severe pulmonary fibrosis. Death of pulmonary epithelial and endothelium cells as a result of apoptosis and increasing oxidative stress can be considered as common symptoms in the mentioned diseases.

International Patent document NO. WO2012142498A2, discloses a method using an efficient amount of MIF inhibitors and a molecule used in treatment and prevention of diseases associated with MIF (including pulmonary diseases). In this study, it is stated that GLP-2 analogues can be used as auxiliary therapeutic agent with MIF inhibitor.

U.S. Pat. No. 8,372,980B2 discloses a molecule (thrombin receptor antagonist) comprised of a himbacine derivative used in treatment of diseases including inflammatory diseases in the lung. In this study, there are compounds combined with the said molecule and reducing such adverse effects for radiation or chemical based toxicity not damaging the non-malignant tissue. One of these is the group which is used against the damage that will be created by radiotherapy and comprises teduglutide.

Australian Patent document NO. AU2013201023A1 discloses a cancer treatment method and a kit developed via inhibition of poly-ADP-ribose polymerase. Certain adverse effects have been avoided by applying nitrobenzamide used in combination with antineoplastic agents in the said study. One of the said antineoplastic agent groups is anti-mucositis agents, and there is also teduglutide in this group.

International Patent document NO. WO041779A2 discloses a treatment method which inhibits the apoptosis caused by chemotherapy and triggers the vitality of the cell. In this study, caspase mediated cell death pathway is inhibited. In first stage (pretreatment), h[GLY2]-GLP2 used as GLP-2 receptor activator is administered on a daily in certain periods. Following these applications, chemotherapeutic agents are used. By means of the antiapoptotic effect of GLP-2, bacterial infection caused by cytotoxicity and chemotherapeutic agent is reduced, and thus the rats are protected from the adverse effects of the chemotherapy.

United States Patent document NO. US20090011987A1 discloses a GLP-2 compound used in treatment of ischemia/reperfiision damages. In the study, the term GLP-2 compound includes any derivative of GLP-2 and its antagonists. Additionally, it is disclosed that the mentioned treatment is used in secondary organ damages caused by ischemia/reperfusion including lung. The use of GLP-2 compound is associated with the inhibition of protein synthesis.

Today, teduglutide (trade name Gattex and Revestive) is used as active substance for the treatment of short bowel syndrome disease. Teduglutide provides treatment by triggering cell proliferation in mucosa.

Teduglutide polypeptide presented in patent documents mentioned above is not used for therapeutic purpose in cancer or pulmonary diseases; it is used in combination with active ingredients used for eliminating adverse effects and as an auxiliary ingredient. There is no drug present wherein teduglutide polypeptide is used as active ingredient for therapeutic purpose in various cancer types and pulmonary diseases.

### SUMMARY OF THE INVENTION

The objective of the present invention is to provide the use of GLP-2 analogues as a therapeutic and protective agent with antiapoptotic, antioxidant and anti-inflammatory effect in pulmonary diseases.

Another objective of the present invention is to provide a use of GLP-2 analogues as a therapeutic and protective agent in pulmonary diseases caused by pulmonary epithelial and/or endothelial cell damages.

A further objective of the present invention is to provide use of the GLP-2 analogues in production of a specific drug to prevent pulmonary diseases caused by epithelial, endothelial and/or mesenchymal cell damage based on oxidative stress, inflammation and apoptosis.

Another objective of the present invention is to provide a therapeutic and preventive method for epithelial, endothelial and/or mesenchymal cell damages caused by oxidative stress, inflammation and apoptosis with a drug comprising GLP-2 analogue.

### DETAILED DESCRIPTION OF THE INVENTION

“Use of GLP-2 Analogues in Pulmonary Diseases For Therapeutic Purposes” developed to fulfill the objectives of the present invention is illustrated in the accompanying figures, wherein

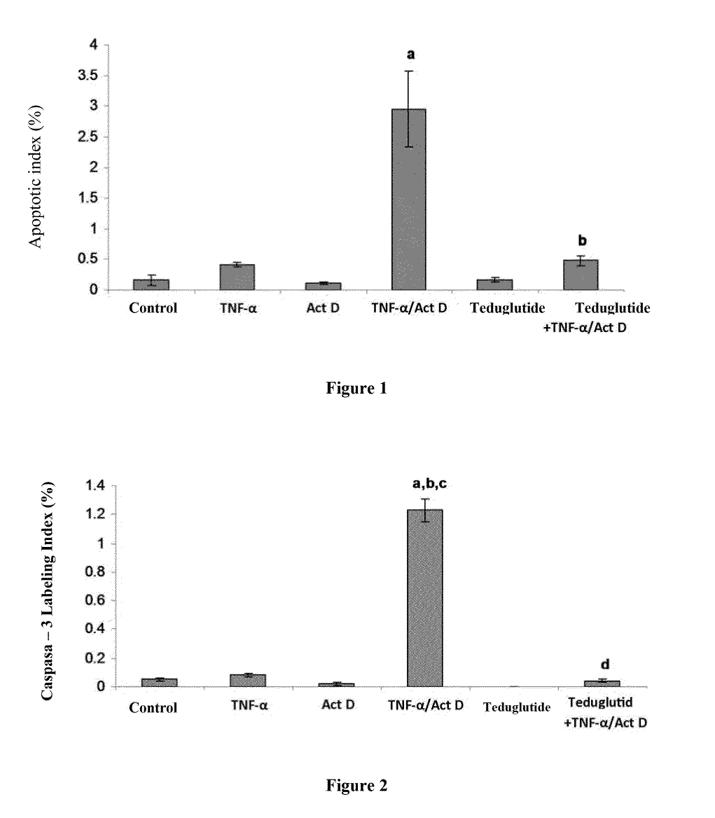

FIG. 1 is the view of apoptotic index (%) in alveolar areas in mice. (Control: 0.16±0.09, TNF-α (TNF-alpha): 0.41±0.04, Act D: 0.11±0.02, TNF-α/Act D: 2.94±0.62, Teduglutide: 0.17±0.04, Teduglutide+TNF-α/Act D: 0.47±0.08. ^{a}P<0.05 relative to the control group, ^{b}P<0.01 relative to the group TNF-α/Act D).

FIG. 2 is the view of caspase-3 index (%) in alveolar areas in mice. (Control: 0.05±0.01, TNF-α: 0.08±0.005, Act D: 0.02±0.005. TNF-α/Act D: 1.23±0.08, Teduglutide: 0.00±0.00, Teduglutide+TNF-α/Act D: 0.04±0.004. ^{a}P<0.05 relative to the control group, ^{b}P<0.05 relative to the group TNF-α, ^{c}P<0.05 relative to group Act D, ^{d}P<0.05 relative to group TNF-α/Act D).

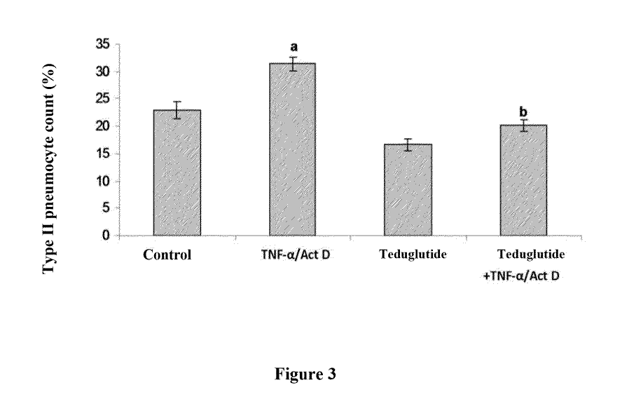

FIG. 3 is the quantitative evaluation of type II pneumocytes in alveolar epithelium of mouse lung. (Control: 22.97±1.54, TNF-α/Act D: 31.48±1.25, Teduglutide: 16.65±1.13, Teduglutide+TNF-α/Act D: 20.13±1.00. ^{a}P<0.05 relative to the control group, ^{b}p<0.05 relative to the group TNF-α/Act D).

The invention is method for preventing an effect caused by oxidative stress, inflammation and/or apoptosis by applying GLP-2 analogue to an organism in an efficient amount.

The invention is use of GLP-2 analogue in an efficient amount for the production of a specific drug to prevent an effect which is caused by oxidative stress, inflammation and/or apoptosis in an organism.

The term “organism” refers to vertebrate with pulmonary respiration. In a preferred embodiment of the invention, the organism is a mammalian. In another preferred embodiment of the invention, the organism is human.

The expression “an efficient amount” in the invention is the amount which the GLP-2 analogue provides beneficial effect on the organism to which the inventive drug is administered. The said amount administered to the organism varies according to the state and size of the organism to which the drug is administered, the purpose for which the drug is administered to the organism, and other parameters known in the state of the technology determining an efficient amount. Determining procedure of an efficient amount comprises routine optimization processes within the capabilities of the experts skilled in the technology.

In the present invention, “an effect” caused by oxidative stress, inflammation and/or apoptosis means pulmonary diseases stimulated by epithelial, endothelial and mesenchymal cell apoptosis, oxidative stress and/or inflammation. The present invention enables production a drug providing treatment for at least one of the disease group comprised of acute and chronic pulmhnonary diseases comprising acute respiratory failure syndrome, chronic obstructive pulmonary disease, emphysema, pulmonary fibrosis, pulmonary hypertension, asthma and lung cancer in which pulmonary endothelial and epithelial apoptosis is induced secondarily.

In the present invention, the term “preventing an effect” occurring in the organism refers to treating the effect or protecting the organism by preventing the effect.

Cytokines are the regulators regulating the responses such as infection, immune response, inflammation. The cytokines are named as lymphokines, interleukins and chemokines according to the cells they are secreted, their functions or effect mechanisms. Some cytokines trigger the pathogenesis of diseases as proinflammatory, while some are present in healing process by reducing inflammation as anti-proinflammatory. Interleukin-1 and (IL-1 IL-1), IL-2, IL-3, IL-5, IL-6, IL-8, IL-12, IL-13, IL-17, IL-18, IL-21, IL-23, IL-27, tumor necrosis factor alpha (TNF-alpha), interferon-γ (IFN-γ) and TGF-β can be given as examples for proinflammatory cytokines.

TNF-alpha (Tumor necrosis factor alpha) is multifunctional cytokine, and plays an important role in pathogenesis of many inflammatory pulmonary diseases. They have roles in cell proliferation, survival, immunity, apoptosis and signal mechanism of inflammation. In high concentration, TNF alpha triggers cytotoxicity and cell deaths by stimulating prooxidative and inflammatory processes in lung.

In the present invention, the pulmonary diseases stimulated with pulmonary endothelial, epithelial and mesenchymal cell apoptosis, oxidative stress and/or inflammation are pulmonary diseases caused by proinflammatory cytokine activity. In the preferred embodiment of the invention, the pulmonary diseases stimulated with pulmonary endothelial, epithelial and mesenchymal cell apoptosis, oxidative stress and/or inflammation are TNF-alpha mediated pulmonary diseases.

The invention provides the use of GLP-2 analogue in prevention of TNF-alpha mediated pulmonary diseases. The invention is to use GLP-2 analogue in an efficient amount for the production of a drug in prevention of TNF-alpha mediated pulmonary diseases.

Human GLP-2 (glucagon like peptide 2) peptide is comprised of 33 amino acid sequence (SEQUENCE ID No. 1). In the present invention, “GLP-2 analogue” is GLP-2 agonist or GLP-2 derivative in other words. GLP-2 analogue is a biologically active peptide which is similar to GLP-2 peptide defined with SEQUENCE ID NO. 1 sequence and has GLP-2 activity. In the preferred embodiment of the invention, GLP-2 analogue is comprised of adding at least one amino acid, and/or removing, and/or changing, and/or adding at least one amino acid to NH₃ end or COO end in Sequence ID No. 1 sequence. GLP-2 analogue peptide sequence is at least 70% similar to SEQUENCE ID NO. 1.

In the present invention, GLP-2 analogue is used as a therapeutic and protective (profilactive) agent exhibiting at least 80% effect as antiapoptotic, antioxidant and antiinflammatory agent in pulmonary diseases. It is used in pulmonary diseases wherein pulmonary epithelial, endothelial and/or mesenchymal cell apoptosis, oxidative stress and inflammation is stimulated. Preferably, it is used in pulmonary diseases wherein pulmonary epithelial, endothelial and/or mesenchymal cell apoptosis, oxidative stress and inflammation caused by TNF-alpha is stimulated. Pulmonary epithelium cells are preferably type I and type II alveolar epithelial cells. In the preferred embodiment of the invention, pulmonary epithelial cells are type II pneumnocyte cells.

In the preferred embodiment of the invention, GLP-2 analogue is teduglutide (h[Gly2]GLP-2, teduglutide). Teduglutide is a polypeptide sequence of 33 amino acid residues (SEQENCE ID NO. 2).

In the present invention, the drug comprising GLP-2 analogue is preferably administered to the organism by injection, that is subcutaneous, intramuscular or intravenous injection or via subdermal infusion pump or via pulmonary delivery. In the preferred embodiment of the invention, the inventive drug is administered to the organism via subcutaneous injection.

In the preferred embodiment of the invention, the drug comprising GLP-2 analogue in an efficient amount is administered in a dose range of approximately 0.0005 mg/kg to 15 mg/kg in a day depending on the weight of the organism to which the drug will be administered via subcutaneous injection. Doses are administered once to four times a day, preferably once to twice a day. The drug comprising GLP-2 analogue with the determined doses and intervals is administered as a protective and therapeutic agent with at least 80% effect.

In the present invention, the daily dose varies according to the drug's way of administration to the organism. The daily dose amount of the drug administered to the organism via uninterrupted infusion is usually less; while the daily dose of the drug administered with another method except for injection is more.

In the preferred embodiment of the invention, the said drug is in a salt form or in form of a pharmaceutical composition with other pharmaceutically acceptable auxiliary ingredients or with suitable carrier systems such as liposome, niosome, nanoparticle, microemulsion, multiemulsion and microsponges in treatment of pulmonary diseases stimulated by pulmonary epithelial, endothelial and/or mesenchymal cell apoptose, oxidative stress and/or inflammation. The pharmaceutical compositions comprising the said invention can be administered to the organism in any form via any method of administration.

Within the framework of these basic concepts, it is possible to develop a wide variety of embodiment of the inventive use of GLP-2 analogues in pulmonary diseases for therapeutic purposes. The invention cannot be limited to the examples described herein and it is essentially according to the claims.

### EXAMPLES

The examples below are included in order to enable the present invention to be understood. Undoubtedly, the experiments related with the present invention should not be interpreted as limiting the invention, and it should be considered that the variations known by the person skilled in the technology on the subject of the invention or the variations to be developed in the future are included in the scope of the invention as described here and disclosed in the claims hereinafter.

### Example 1

### The Medicinal and Therapeutic Effect of Teduglutide on Alveolar Wall

TNF-alpha (Tumor necrosis factor alpha) plays an important role in pathogenesis of many inflammatory pulmonary diseases in experimental studies on the present invention. TNF-alpha has various effects on cell proliferation, survival, apoptosis, immunity and signaling cascades of inflammation. In high concentration, TNF-alpha triggers cytotoxicity and cell deaths by stimulating prooxidative and inflammatory reactions in lung.

Actinomycin D (Act D) is a transcription inhibitor, and it is an anti-neoplastic drug triggering tissue damage via reduction in nucleotides, therefore RNA and protein synthesis. By using Act D with TNF-alpha, cell, tissue, organ and organism are made sensitive to damage, and the cytotoxic effect of TNF-alpha is increased, and apoptotic cell death is triggered.

TNF-alpha and/or Act D was administered to mice in the experiments. Act D is used in order to increase the cytotoxic effect of TNF-alpha in lung tissues. In this way, pulmonary epithelial, endothelial and mesenchymal cells were damaged in mouse lung by stimulating apoptosis, inflammation and oxidative stress. TNF-alpha. Act D and especially the combination of TNF-alpha/Act D cause the damage in alveolar wall of mice lung; it reduces the tissue factor (TF) and sodium-potassium ATPase (Na-K-ATPase) activities while stimulating apoptosis in pulmonary endotheliaL % epithelial cells, myeloperoxidase (MPO) activity in tissue and oxidative stress.

In this study, 8-10 months male BALB/c mice were used. Mice were divided into 6 groups:
Group I (Control group): Mice injected with 0.1% dimethylsulfoxide (DMSO) and phosphate buffered saline (PBS, pH 7.4) via intraperitoneal way.
Group II: Mice injected with 15 μg/kg TNF-alpha (dissolved in PBS) with single dose via intraperitoneal way.
Group III: Mice injected with 800 μg/kg Act D (dissolved in DMSO) with single dose via intraperitoneal way.
Group IV: The mice given with TNF-alpha 2 minutes after Act D is administered in doses and mode of administrations above.
Group V: The mice given with 200 μg/kg teduglutide (dissolved in PBS) for 10 days uninterruptedly in every 12 hours via subcutaneous injection.
Group VI: TNF-alpha was injected to mice to which teduglutide was administered for 10 days as mentioned above, 2 minutes after Act D was administered in 11th day in above doses and mode of administration.

The mice in Group V were cut with cervical dislocation 16.5 hours after the last administration, and the mice in other groups were cut 4.5 hours after the last administration, and the lung samples were taken for microscopic and biochemical analysis.

Some part of the left lung samples taken for histological studies were fixed at room temperature for 24 hours in Bouin solution. The samples were embedded in paraffin after dehydration in alcohol and clearing in xylene. The sections having thickness of 5 μm taken from lungs blocked in paraffin were stained with hematoxilin-eosin (HE) and examined under light microscope.

As a result of the examination, healthy mice lungs exhibited normal tissue structure. TNF-alpha/Act D administration resulted in damage and thinning in some areas of alveolar wall. It was determined that the damage effect was more in lung tissues wherein TNF-alpha and Act D were administered together and partially broken alveolar walls were seen in these samples. It was seen that structural damages were decreased in alveolar epithelium structure in mice treated with teduglutide and TNF-alpha/Act D administered. Furthermore, it was observed that alveoli were in better condition in healthy mice lung tissues treated only with teduglutide.

Alveolar epithelium tissue comprises type I and type II pneumocytes. TNF-alpha triggers apoptosis in bronchial epithelial cells, and pulmonary epithelial, endothelial and mesenchymal cells.

### The Effect of Teduglutide on Apoptosis

TUNEL (Terminal deoxynucleotidyl transferase-mediated dUTP-nick end labeling assay) method was used to identify apoptotic cells in the lung. In this method, left lung sections fixed with 10% neutral formalin for 24 hours were used. Lung sections in thickness of 5 μm were treated with proteinase K (20 ug/ml) following rehydratation, and then endogenous peroxidase activity was blocked with 3% H₂O₂.

Marking step was carried out with commercially available ApopTag Plus Peroxidase kit (Millipore). The reaction in apoptotic cells was developed with 3,32-diaminobenzidine. Following this process, cell nuclei were made visible in staining with methylene green. Mice breast tissue was used as positive control. Instead of Tdt enzyme PBS was used as negative controls. TUNEL positive cells in the lung were analyzed under light microscope at 400× magnification. The apoptotic index value was calculated as a percentage of apoptotic cells in total number of cells.

Apoptosis was identified in pulmonary epithelial cells, pulmonary endothelial cells and mesenchymal cells of connective tissue in the alveolar region by TUNEL method. Microscopic observations showed that apoptotic index in the alveolar region was increased significantly in mice lungs administered with TNF-alpha/Act D when compared with the control group. However no significant change in apoptotic index was observed in mice lungs to which TNF-alpha or Act D or teduglutide is administered alone. Teduglutide significantly reduces apoptotic index in mice given TNF-alpha and Act D (FIGS. 1 and 2).

For the identification of apoptosis in the tissue, as another way, active caspase-3 immunoreactivity was tested. For this purpose, streptavidin-biotin-peroxidase immune method was used. Sections of left lung fixed with neutral formalin were treated with 0.3% triton X-100 for 10 minutes after dewaxing and rehydratation. Following this process, they heated in 10 mM citrate buffer (pH 6.0) for 15 min in a microwave oven. The sections were incubated for 10 minutes with 3% H₂O₂ for blocking of endogenous peroxidase activity. The next steps were performed according to the instructions in Histostain Plus Broad Spectrum Kit (Invitrogen). The sections were incubated with polyclonal rabbit active caspase 3 antibody (1:50 in PBS, Millipore) for 1 hour at room temperature.

Immunoreaction was developed with 3-amino-9-ethylcarbazole. The sections were stained with Mayer's hematoxilin following immunostaining. PBS or nonspecific rabbit IgG primary antibody were used as negative controls.

Quantitative analysis of immunoreactive cells labeled with caspase-3 antibody was made with light microscope at ×400 magnification. For the immunoreactive cell count, 5 randomly selected fields of alveolar areas without bronchioles were examined in the lung sections of each mouse. The labeling index for caspase-3 was calculated as a percentage of immunoreactive cells in the total number of cells counted per section.

The number of active caspase-3 immunoreactive cells in the alveolar area increased significantly in individuals to which TNF-alpha/Act D was administered relative to control TNF-alpha or Act D groups. This number decreased significantly in individuals previously treated with teduglutide to which TNF-alpha/Act D was administered. It was identified with TUNEL method that the apoptotic index in alveolar area was higher in individuals to which TNF-alpha/Act D was administered than the individuals to which only TNF-alpha or only Act D was administered. These data were confirmed with active caspase-3 marker. TNT-alpha/Act D triggers apoptosis in lung alveolar epithelium, pulmonary endotheliumn and mesenchymal cells. Increase in apoptotic index in alveolar area is an important factor causing lung damage.

### The Effect of Teduglutide on Cell Proliferation in Alveolar Region

Strep-ABC immunostaining method was used in order to determine proliferative cells in the alveolar area. The steps applied for immunohistochemical method were the same with the method used to determine caspase-3 immunoreactivity. Being different, in step of primary antibody administration, the lung sections were incubated with rabbit Ki-67, a proliferation marker, (1:100 diluted) for one hour at room temperature. Furthermore, since it is known that type II pneumocytes give new ones to replace type I and type II pneumocytes which die or lose function in the damaged alveolar epithelium, the number of type II pneumocytes in the alveolar area were analyzed in the experimental groups. The above mentioned Strep-ABC method was used for this process. In primary antibody step, hmg sections were incubated overnight at 4° C. with rabbit Pro-SPC (Type II pneumocyte marker, Milipore, 1:1000 dilution). Proliferation index and number of type II pneumocytes in the alveolar area were calculated as ratio of immunoreactive cells in total number of cells, and the value was expressed as %. Counts were performed in 5 different randomly selected fields for each animal's lung section at ×400 microscopic magnification.

The increase in apoptotic index in alveolar area of mice to which TNF-alpha/Act D was administered, accompanied with increase in type II pneumocyte. The increase in type II pneumocyte number in the lungs is an indicator of decrease in type I pneumocytes. This situation is the repair response of alveolar epithelium against damage. Type II pneumocytes increasing in alveolar epithelium number will regenerate alveolar epithelium cells which were damaged and died with apoptosis. Therefore, the damage (thinning and breaking) on the alveolar epithelium and alveolar wall is directly related with pneumocyte, endothelial cells and mesenchymal cells.

In individuals treated with teduglutide before TNF-alpha/Act D administration, a significant decrease in type II pneumocyte number was observed. According to this, teduglutide prevents directly the apoptosis in alveolar area and protects the alveolar area against damage instead of inducing on type II pneumnocyte proliferation.

### Labeling GLP-2 Receptors (GLP-2R) in the Lung

Daughter sections from serial lung sections were incubated with rabbit antibody against GLP-2R (Chemicon 1:100 diluted) for 1 hour at room temperature and with rabbit antibody against Pro-SPC (Milipore, 1:1000 diluted) overnight at 4 C.° by the above mentioned Strep-ABC immunostaining method. It was observed that type II pneumocyte and some mesenchymal cells of connective tissue have GLP-2 receptors. Teduglutide can affect their biological behaviors directly by connecting to GLP-2 receptor localized in these cells. Taking into consideration the apoptotic index and the active caspase-3 immunoreactivity between the groups, teduglutide directly protects the type II pnemnocytes, which is one of target cells of teduglutide, against apoptotic cell death mediated by TNF-alpha/Act D. (FIG. 3)

### Example 3

### The Effect of Teduglutide on Oxidative Stress and Inflammation

For biochemical analysis, right lungs taken from the mice were homogenized in cold 0.9% NaCl to make 10% (w/v) homogenate. The supernatants obtained from centrifuged homogenates were used for GSH (glutathione), lipid peroxidation (LPO) and enzyme analyses.

The GSH levels of lung homogenates were determined according to Beutler method (1975) by using Ellman's reagent, LPO levels were determined according to the method of Ledwozyw et al. (1986), catalase (CAT) and superoxide dismutase (SOD) activities were determined according to Aebi (1984) and Mylroie (Mylorie et al., 1986), respectively, glutathione peroxidase (GPx) activity was determined according to Paglia and Valentine (1967) method modified by Wendel (1981). The myeloperoxidase activity (MPO) in lung tissues was determined according to Wei and Frenkel (1991) method, Na⁺K⁺ATPase activity was determined according to method by Ridderstap and Bonting (1969), xanthine oxidase (XO) activity was determined according to Corte and Stirpe (1968) method apart from several modifications, tissue factor (TF) activity in lung tissue was determined with Quick's one-stage method (1976) using normal plasma, and the protein level in tissue was determined with Lowry method (1951) using standard bovine serum albumin.

Glutathione has an important role as reductant in oxidation reduction process and it has also function in detoxification. In a healthy individual, GSH is important in terms of reducing the effects of free radicals. Tissue damages caused by oxidative stress are generally related with the decrease in GSH level in the tissue. However oxidative stress can increase GSH synthesis in the endothelial cells. (Table 1)

Biochemically, the GSH levels in lung tissues were looked at, and it was seen that GSH level increased significantly in subjects to which TNF-alpha, Act D, teduglutide, TNF-alpha/Act D was administered relative to the control group. However, it was observed that GSH level in individuals treated with teduglutide and TNF-alpha/Act D decreased significantly when compared to animals administered by TNF-alpha/Act D. The increase in GSH level in lung tissue occurred as a result of the acceleration in GSH synthesis as a cellular defense mechanism against toxic stimulations.

Oxidative stress causes the production of reactive oxygen species (ROS) such as superoxide anion, hydroxyl radical, H₂O₂ and singlet oxygen. SOD converts superoxide into H₂O₂. H₂O₂ transforms into water and molecular oxygen via CAT and GPx. However, H₂O₂ reacts with iron and produces hydroxyl radical which causes the generation of lipid peroxides and other organic radicals.

TNF-alpha increases ROS production and causes cellular damage also in human endothelial cells as well as in mouse lung and liver.

In LPO process, fatty acids are converted into secondary metabolites such as 4-hydroxylalkenal and malonaldehyde. Malonaldehyde metabolite is used as LPO indicator.

Biochemically, the LPO levels in lung tissues were looked at, and it was seen that LPO level increased significantly in subjects to which TNF-alpha, Act D, teduglutide, TNF-alpha/Act D was administered relative to the control group. However, it was observed that LPO level in individuals treated with teduglutide and TNF-alpha/Act D decreased significantly when it is compared with mice to which TNF-alpha/Act D was administered. Teduglutide significantly prevents increase in LPO level. (Table 1)

TABLE 1
GSH and LPO levels in lung tissues

| | GSH | LPO |
|---|---|---|
| | (nmol GSH/ | (nmol MDA/ |
| Groups | mg protein)* | mg protein)* |
| Control | 6.27 ± 3.00 | 2.72 ± 1.07 |
| TNF-α | 10.16 ± 2.66^{a} | 4.51 ± 0.50^{b} |
| Act D | 18.79 ± 8.78^{b} | 5.10 ± 1.78^{b} |
| TNF-α/Act D | 48.76 ± 5.49^{c} | 4.94 ± 1.74^{b} |
| Teduglutide | 43.67 ± 8.25^{c} | 5.31 ± 1.71^{b} |
| Teduglutide + | 29.40 ± 3.59^{d} | 2.97 ± 0.63^{e} |
| TNF-α/Act D | | |
| P_{ANOVA} | 0.0001 | 0.0001 |

*Mean ± SD
^{a}P < 0.05 versus control group
^{b}P < 0.005 versus control group
^{c}P < 0.0001 versus control group
^{d}P < 0.0001 versus TNF-α/Act D group
^{e}P < 0.005 versus TNF-α/Act D group

SOD activity gives information about superoxide production; CAT and GPx activities give information about H₂O₂ cycle. TNF-alpha contributes to the generation of oxidative stress in the cell by formation of superoxide anions and hydroxyl radicals.

CAT, SOD, GPx and MPO activities were analyzed in lung samples, it was seen that MPO activity, which is considered as an indicator or neutrophil infiltration and tissue inflammation, increased significantly in individuals to which TNF-alpha, Act D, teduglutide, TNF-alpha/Act D was administered relative to the control group. However, it was observed that MPO activity in individuals pretreated with teduglutide to which TNF-alpha/Act D is given decreased significantly when compared to TNF-alpha/Act D group. (Table 2)

TABLE 2
CAT, SOD, GPx, MPO activities in lung tissues

| | CAT | SOD | GPₓ | MPO |
|---|---|---|---|---|
| | (U/mg | (U/mg | (U/g | (mU/g |
| Groups | protein)* | protein)* | protein)* | tissue)* |
| Control | 37.23 ± 5.13 | 3.33 ± 1.85 | 262.05 ± 38.49 | 37.85 ± 10.11 |
| TKF-α | 42.44 ± 5.79^{a} | 6.99 ± 2.57^{f} | 453.86 ± 52.35^{c} | 193.80 ± 31.28^{c} |
| Act D | 29.76 ± 4.34^{b} | 8.21 ± 2.29^{g} | 473.28 ± 78.26^{c} | 109.01 ± 28.83^{c} |
| TNF-α/Act D | 56.83 ± 7.78^{c} | 13.47 ± 3.59^{c} | 529.00 ± 68.29^{c} | 241.04 ± 47.92^{c} |
| Teduglutide | 44.74 ± 7.52^{d} | 7.36 ± 1.28^{c} | 491.56 ± 171.49^{b} | 88.93 ± 30.32^{g} |
| Teduglutide + | 31.47 ± 3.24^{e} | 6.82 ± 1.67^{e} | 392.90 ± 85.30^{h} | 69.21 ± 23.94^{e} |
| TNF-α/Act D | | | | |
| P_{ANOVA} | 0.0001 | 0.0001 | 0.0001 | 0.0001 |

*Mean ± SD
^{a}P > 0.05 versus control group
^{b}P < 0.01 versus control group
^{c}P < 0.0001 versus control group
^{d}P < 0.05 versus control group
^{e}P < 0.0001 versus TNF-α/Act D group
^{f}P < 0.01 versus control group
^{g}P < 0.001 versus control group
^{h}P < 0.01 versus TNF-α/Act D group

In this study, administration of TNF-alpha, Act D, TNF-alpha/Act D, and teduglutide to mice induced LPO and ROS production as well as increase in SOD, CAT, GPx activities in lung. Additionally, increased LPO level in the lung upon administration of TNF-alpha/Act D does not decrease despite of the increase in GSH level and in SOD, CAT, GPx activities. The reason for this is that ROS and LPO production may induce the antioxidant intracellular defense mechanism to operate and thus it may stimulate antioxidant enzyme expression. ROS and LPO productions triggered with TNTF-alpha/Act D increases the antioxidant enzyme capacity. Therefore the activated antioxidant system prevents structural damage in the alveolar wall. With teduglutide and TNT-alpha/Act D treatment, GSH and LPO levels and CAT, SOD and GPx activities in the tissues are decreased when compared to TNF-alpha/Act D group. However, no decrease in GSH level and SOD, GPx activities is seen in the group given teduglutide and TNF-alpha/Act D relative to control group despite the LPO decrease with teduglutide pre-treatment. Pretreatment with teduglutide prevented the oxidative tissue damage stimulated with TNF-alpha/Act D and regressed the LPO levels to normal.

It is known that oxidative stress triggers certain signal cascades resulted in apoptosis. Teduglutide prevents TNF-alpha/Act D-induced apoptosis and oxidative stress related with LPO.

MPO indirectly causes neutrophil infiltration during an inflammatory effect. Inflammatory cells secrete ROS and various cytokines (such as TNF-alpha) and trigger inflammatory reactions in this way. Therefore, tissue damage is formed with the epithelial cell deaths.

In this study, a significant increase in MPO activity was observed with TNF-alpha, Act D and TNF-alpha/Act D combination. With the teduglutide pretreatment, an effective decrease in MPO activity was seen in individuals to which TNF-alpha/Act D was administered. Teduglutide plays a protective role against inflammatory lung damage stimulated by TNF-alpha/Act D.

Na⁺K⁺ATPase is an enzyme functioning in cellular carrying and localized in membrane. It is highly sensitive to LPO and other free radical reactions. The decrease in Na+K+ATPase activity is associated with deterioration of cell transport and indirect indicator of membrane damage. With the increasing LPO levels in the tissue, the decrease in Na+K+ATPase activity may result in losses of physiological functions in the lung. (Table 3)

TF is an important coagulation factor. It is present in brain and lung in high concentration, while its activity is highest in the lung. Its activity is changed with the membrane composition alterations or LPO caused by oxidative stress. Thromboplastin level increases in the tissue upon decrease in TF activity and thus cellular damage is formed. The decreased TF activity mediated by oxidative stress generated tissue damage in mice to which TNF-alpha, Act D, TNF-alpha/Act D is administered. Teduglutide increases the TF activity, exhibits protective effect against lung damage stimulated by TNF-alpha/Act D and triggers tissue healing. (Table 3)

In tissues subjected to metabolic stress such as inflammation, hypoxia and ischemia, xanthine dehydrogenase enzyme is converted into xanthine oxidase (XO) during ATP degradation. Xanthine oxidase also triggers oxidative damage by being the main source of reactive oxygen species and contributing to production of superoxide radical (O₂⁻) in biological systems. The increase in pulmonary XO activity is an indicator of ATP degradation and it is associated with the increase in oxidative damage in the tissue in individuals to which TNF-alpha, Act D, TNF-alpha/Act D were administered. Teduglutide plays a protective role against lung damage triggered by TNF-alpha/Act D, it provides LPO inhibition and decreases the XO activity. (Table 3)

It was seen that XO activity increased significantly in subjects to which TNF-alpha, Act D, Teduglutide. TNF-alpha/Act D was administered relative to the control group. However, it was observed that XO activity in animals treated with teduglutide and TNF-alpha/Act D decreased significantly when it is compared with mice to which TNF-alpha/Act D was administered.

TABLE 3
Na⁺K⁺ATPase, XO and TF activities in lung tissues

| | Na⁺K⁺ATPase | XO | |
|---|---|---|---|
| | (nmol Pᵢ/mg | (U/mg | TF |
| Groups | protein/h)* | protein)* | (sec)* |
| Control | 9.15 ± 2.45 | 7.76 ± 3.76 | 52.71 ± 14.84 |
| TNF-α | 3.96 ± 0.71^{a} | 21.53 ± 3.28^{a} | 49.08 ± 10.40^{d} |
| Act D | 3.63 ± 1.13^{a} | 24.37 ± 4.25^{a} | 50.50 ± 8.06^{d} |
| TNF-α/Act D | 0.79 ± 0.30^{a} | 21.44 ± 3.65^{a} | 49.08 ± 10.65^{d} |
| Tedoglutide | 6.46 ± 1.13^{a} | 17.46 ± 3.16^{a} | 47.28 ± 9.91^{d} |
| Teduglutide + | 9.54 ± 1.67^{b} | 6.91 ± 1.66^{c} | 60.35 ± 14.31^{e} |
| TNF-α/Act D | | | |
| P_{ANOVA} | 0.0001 | 0.0001 | 0.382 |

*Mean ± SD
^{a}P < 0.0001 versus control group
^{b}P< 0.001 versus TNF-α/Act D group
^{c}P < 0.0001 versus TNF-α/Act D group
^{d}P > 0.05 versus control group
^{e}P > 0.05 versus TNF-α/Act D group

The references cited in the description are as follows:

### REFERENCES

JEPPESEN, P. B., HARTMANN. B., THULESEN, J., GRAFF, J., LOHMANN, J., HANSEN, B. S., TOFTENG, F., POLrLSEN, S. S., MADSEN, J. L., HOLST; J. J., MORTENSEN, P. B., 2001, Glucagon-Like Peptide-2 Improves Nutrient Absorption and Nutritional Status in Short-Bowel Patients with No Colon, Gastroenterology, 120, 806-815.
DRUCKER. DJ., EHRLICH, P., ASA, S. L., BRUBAKER, P. L., 1996, Induction of Intestinal Epithelial Proliferation by Glucagon-Like Peptide-2, Proceedings of the National Academy Sciences, 3, 7911-7916.
ESTALL, J. L., DRUCKER, D. J., 2003, Dual Regulation of Cell Proliferation and Survival via Activation of Glucagon-Like Peptide-2 Receptor Signaling, Journal of Nutrition, 133, 3708-3711.
ESTALL, J. L., DRUCKER, D. J., 2005, Tales beyond the Crypt: Glucagon-Like Peptide-2 and Cytoprotection in the Intestinal Mucosa, Endocrinology, 146 (1), 19-21.
BOUSHEY, R. P., YUSTA, B., DRUCKER, D. J., 2001, Glucagon-Like Peptide (GLP)-2 Reduces Chemotherapy-Associated Mortality and Enhances Cell Survival in Cells Expressing a Transfected GLP-2 Receptor, Cancer Research, 61, 687-693.
PRASAD, R., ALAVL K., SCHWARTZ, M. Z., 2000, Glucagon-like Peptide-2 Analogue Ehances Intestinal Mucosal Mass and Absorptive Function after Ischemia-reperfusion Injury, Journal of Pediatric Surgery, 35, 1537-1539.
L'HEUREUX, M., BRUBAKER, P. L., 2003, Glucagon-Like Peptide-2 and Conunon Therapeutics in a Murine Model of Ulcerative Colitis, Journal of Pharmacology and Experimental Therapeutics, 306 (1), 347-354.
JEPPESEN PB, LUND P, GOTTSCHALCK IB, NIELSEN HB, HOLST JJ, MORTENSEN J, POULSEN SS, QUISTORFF B, MORTENSEN PB. Short bowel patients treated for two years with glucagon-like Peptide 2: effects on intestinal morphology and absorption, renal fimunction, bone and body composition, and muscle function. Gastroenterology Research and Practice, 2009; 2009:616054. (Volume 2009, Article ID 616054, 12 pages doi:10.1155/2009/616054)
O'KEEFE SJ, JEPPESEN PB, GLROY R. PERTKIEWICZ M, ALLARD JP, MESSING B. Safety and Efficacy of Teduglutide After 52 Weeks of Treatment in Patients With Short Bowel Syndrome Intestinal Failure. Clinical Gastroenterology and Hepatology, 2013 July; 11(7):815-823.
YUSTA, B., HUANG, L., MUNROE, D., WOLFF, G., FANTASKE, R., SHARMA, S., DEMCHYSHYN, L., ASA, S. L., DRUCKER, D. J., 2000, Enteroendocrine localization of GLP-2 Receptor Expression in Humans and Rodents, Gastroenterology, 119, 744-755.
BJERKNES, M., CHENG, H., 2001, Modulation of specific intestinal epithelial progenitors by enteric neurons, Proceedings of the National Academy of Sciences of the United States of America, 12497-12502.
LOVSHIN, J., DRUCKER. D. J., 2000. New Frontiers in the Biology of GLP-2, Regulatory Peptides, 90, 27-32.
DE HEER J, PEDERSEN J, ORSKOV C, HOLST JJ. The alpha cell expresses glucagon-like peptide-2 receptors and glucagon-like peptide-2 stimulates glucagon secretion from the rat pancreas. Diabetologia, 2007:50(10):2135-42.
ANGELONE T, FILICE E, QUiNTIER A M, IMBROGNO S, AMODIO N, PASQUA T, PELLEGRINO D, MULE F, CERRA MC. Receptor identification and physiological characterisation of glucagon-like peptide-2 in the rat heart. Nutr Metab Cardiovasc Dis. 2012:22(6):486-94.

1-18. (canceled)

19. A method of treating pulmonary disease in a subject, said method comprising administering a therapeutically effective amount of a GLP-2 analogue to said subject.

20. The method of claim 19, wherein said subject is a human subject.

21. The method of claim 19, wherein said pulmonary disease is caused by (i) apoptosis of the pulmonary epithelia, endothelial, and/or mesenchymal cells; (ii) oxidative stress; and/or (iii) inflammation.

22. The method of claim 19, wherein said pulmonary disease is acute or chronic pulmonary disease.

23. The method of claim 22, wherein said pulmonary disease is selected from the group consisting of: acute respiratory failure syndrome, chronic obstructive pulmonary disease, emphysema, pulmonary fibrosis, pulmonary hypertension, and interstitial pulmonary diseases.

24. The method of claim 22, wherein said pulmonary disease is selected from asthma or lung cancer, wherein pulmonary epithelial, endothelial, and mesenchymal cell apoptosis is induced secondarily.

25. The method of claim 22, wherein said disease is characterized by proinflammatory cytokine activity.

26. The method of claim 25, wherein said proinflammatory cytokine activity is TNF-alpha activity.

27. The method of claim 19, wherein said GLP-2 analogue is of a GLP-2 derivative created by: adding at least one amino-acid into a polypeptide having the sequence of SEQ ID NO:1, adding and removing at least one ammo acid to a polypeptide having the sequence of SEQ ID NO:1, changing at least one amino acid in a polypeptide having the sequence of SEQ ID NO:1, or adding at least one amino acid onto the N or C terminus of a protein having the sequence of SEQ ID NO:1.

28. The method of claim 27, wherein said GLP-2 analogue is a biologically active peptide which is similar to GLP-2 peptide having the sequence of SEQ ID NO:1 and has GLP-2 activity.

29. The method of claim 28, wherein the sequence of said GLP-2 analogue has at least 70% identity to SEQ ID NO:1.

30. The method of claim 29, wherein said GLP-2 analogue is a therapeutic and protective agent having anti-apoptotic, antioxidant, and/or anti-inflammatory activity in TNF-alpha originated pulmonary diseases.

31. The method of claim 19, wherein said GLP-2 analogue is teduglutide.

32. The method of claim 19, wherein said GLP-2 analogue is administered by a method selected from the group consisting of: subcutaneous injection, intramuscular injection, intravenous injection, subdermal infusion pump, and pulmonary delivery.

33. The method of claim 32, wherein said GLP-2 analogue is administered by subcutaneous injection.

34. The method of claim 19, wherein said GLP-2 analogue is administered in a dosage of approximately 0.0005 mg/kg per day to 15 mg/kg per day.

35. The method of claim 34, wherein said GLP-2 analogue is administered once per day to four times per day.

36. The method of claim 35, wherein said GLP-2 analogue is administered once or twice per day.

37. The method of claim 19, wherein said GLP-2 analogue is formulated with a carrier and/or pharmaceutically acceptable inactive ingredients.

38. The method of claim 19, wherein said method consists of administering a therapeutically effective amount of a GLP-2 analog to said subject.


SEQUENCE LISTING (2 sequences)

SEQ ID NO: 1 - protein, 32 aa, Homo sapiens
HADGSFSDEMNTILDNLAARDFINWLIQTKIT

SEQ ID NO: 2 - protein, 32 aa, Artificial Sequence
  Synthetic Construct
HGDGSFSDEMNTILDNLAARDFINWLIQTKIT